(19) United States
(12) Patent Application Publication
(10) Pub. No.: US 2009/0107868 A1
(43) Pub. Date: Apr. 30, 2009

(54) Discreet container for disposable undergarments

(76) Inventor: Madeleine Mercier, Austin, TX (US)

Correspondence Address: FORTKORT & HOUSTON P.C., 9442 N. CAPITAL OF TEXAS HIGHWAY, ARBORETUM PLAZA ONE, SUITE 500, AUSTIN, TX, 78759, US

(21) Appl. No.: 12/290,289

(22) Filed: Oct. 29, 2008

Related U.S. Application Data
(60) Provisional application No. 61/001,133, filed on Oct. 30, 2007.

(51) Int. Cl. B65B 13/20; A61B 19/02; B65D 25/22

(52) U.S. Cl. 206/438; 220/751; 221/278; 53/438

(57) ABSTRACT

A device (101) is provided which comprises a frangible, pressurized container (103), and an undergarment (105) disposed and said container.

### CROSS-REFERENCE TO RELATED APPLICATION

This application claims the benefit of priority from U.S. Provisional Application No. 61/001,133, filed Oct. 30, 2007, having the same title, and having the same inventor, and which is incorporated herein by reference in its entirety.

### FIELD OF THE DISCLOSURE

The present disclosure relates generally to consumer products, and more particularly to discreet containers for disposable undergarments.

### BACKGROUND OF THE DISCLOSURE

Disposable undergarments are currently a necessity for millions of Americans. In addition to infants, for whom diapers and training pants are a normal part of childhood development, a significant portion of the adult population also requires disposable undergarments from time to time to deal with menstrual spotting, incontinence, or various medical conditions. In addition, a need for disposable undergarments may arise due to the need to unexpectedly spend a night away from home as may occur, for example, as a result of business or social developments, or on camping trips or other events.

Ideally, a person in need of disposable undergarments should have them readily available, since the need for these products is often unpredictable. However, the social stigma attached to the need for, or use of, such products by adults has made many adult consumers reluctant to carry these products on their persons or to store them in places where they are readily accessible. This is due, for example, to the concern that such products may be discovered by others inadvertently. There is thus a need in the art for a product which overcomes these difficulties. These and other needs may be met by the products and methodologies disclosed herein, and hereinafter described.

### BRIEF DESCRIPTION OF THE DRAWINGS

FIG. 1 is an illustration of a first embodiment of a portable container of the type described herein which contains a disposable undergarment, and which has been adapted to be secured to a keychain.

FIG. 2 is an illustration of the container of FIG. 1 depicting the interior thereof.

FIG. 3 is an illustration of the container of FIG. 1 depicting the container after the seal has been ruptured and the undergarment removed.

FIG. 4 is an illustration of the undergarment of the container of FIG. 1 shown in a highly compressed state.

FIG. 5 is an illustration of the undergarment of the container of FIG. 1 shown in an uncompressed state.

FIG. 6 is an illustration of a second embodiment of a portable container of the type described herein which contains a disposable undergarment.

FIG. 7 is an illustration of the container of FIG. 6 depicting the container after the seal has been ruptured and the undergarment removed.

FIG. 8 is an illustration of a conventional tampon for comparison to the embodiment of FIG. 6.

FIG. 9 is an illustration of a second embodiment of a portable container of the type described herein which contains a disposable undergarment.

FIG. 10 is an illustration of the container of FIG. 9 depicting the container after the seal has been ruptured and the undergarment removed.

### SUMMARY OF THE DISCLOSURE

In one aspect, a device is provided which comprises a frangible, pressurized container having an undergarment disposed therein.

In another aspect, a device is provided which comprises (a) a pressurized container having a wall which includes at least one helical strip, wherein the edges of said at least one strip are secured together with a suitable adhesive; and (b) an undergarment disposed in said container.

In a further aspect, a method for making a device is provided which comprises (a) providing a container; (b) compressing an undergarment through the application of force; (c) placing the undergarment into the container; and (d) charging the container with a positive pressure of gas.

### DETAILED DESCRIPTION

It has now been found that aforementioned needs may be overcome through the provision of one or more disposable undergarments in a portable container which is not indicative of its contents. The undergarments are preferably in a highly compressed state such that the container may be of manageable size and weight. The container is also preferably slightly pressurized and contains a frangible wall so that the seal of the container may be readily broken, as by firmly tapping the frangible wall against the edge of a countertop or other suitable surface. Preferably, the container is designed so that, when the seal is broken, the undergarment(s) may be readily removed from it.

The storage of undergarments in a pressurized container is advantageous in that, when the undergarments are removed from the container, the expanding gases within the container help to fluff up the undergarments, thus making them less compressed and softer to the touch than would be the case if the container was vacuum packed. In addition, the interior of the container may be charged with a suitable fragrance such that the undergarments are pleasantly scented when they are removed from the container. This fragrance will typically be dispersed into the air by the expanding gases from within the container when the seal to the container is broken, thereby making use of the product more pleasant and importing a general sense of cleanliness to the user.

Moreover, because the container is preferably designed to not be indicative of its contents, the user may comfortably carry the product on their person or store it in a place where it is readily accessible, without having to worry about potential embarrassment if the product is discovered. However, it should be appreciated that, though suitable design (including, for example, the selection of interesting patterns or colors), the container may be fashioned so that the product is interesting, fun and even fashionable to use.

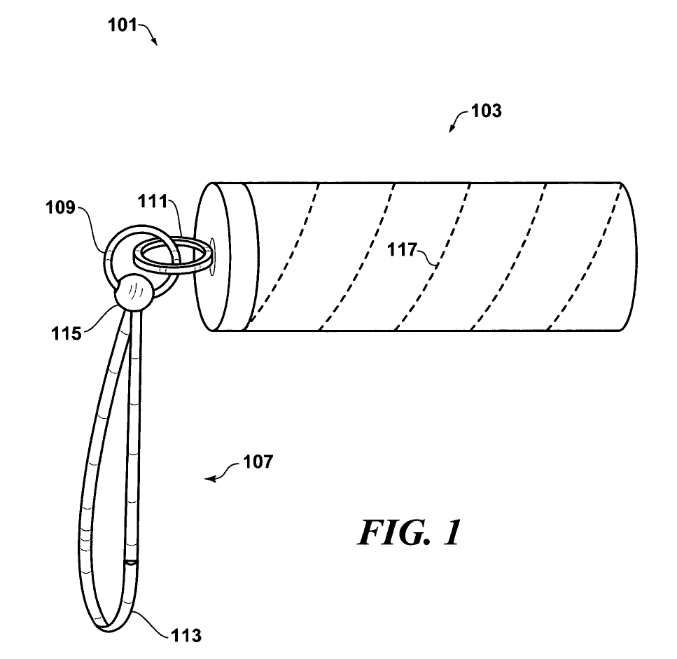

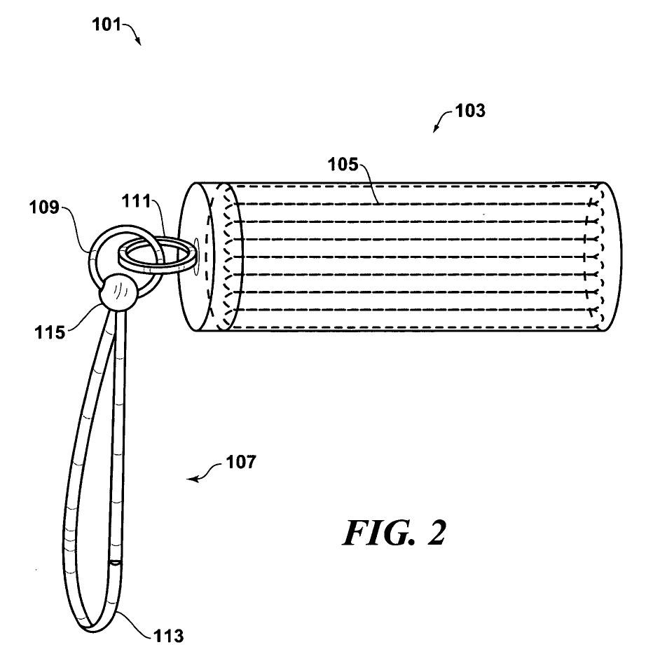

Referring now to FIG. 1, a first particular, non-limiting embodiment of a device made in accordance with the teachings herein is depicted. The device 101 in this particular embodiment comprises a pressurized container 103 which houses a disposable undergarment 105 (see FIG. 2) and which is attached to a keychain 107. The walls of the container 103 comprise a helical strip of cardboard or another suitable material whose edges are glued or adhered together to form a frangible seal 117.

The keychain 107 in this embodiment comprises a hoop 109 which is attached to a ring 111 mounted on one side of the container 103, and terminates in a clasp 113 which can be releasably attached to a set of keys (not shown), to a belt loop, or to another suitable surface. The keychain also comprises a swivel 115 which permits rotation of the keychain about the hoop 109. Of course, it will be appreciated that key chains of various designs and other types of fastening devices may be substituted for the key chain 107 of FIG. 1.

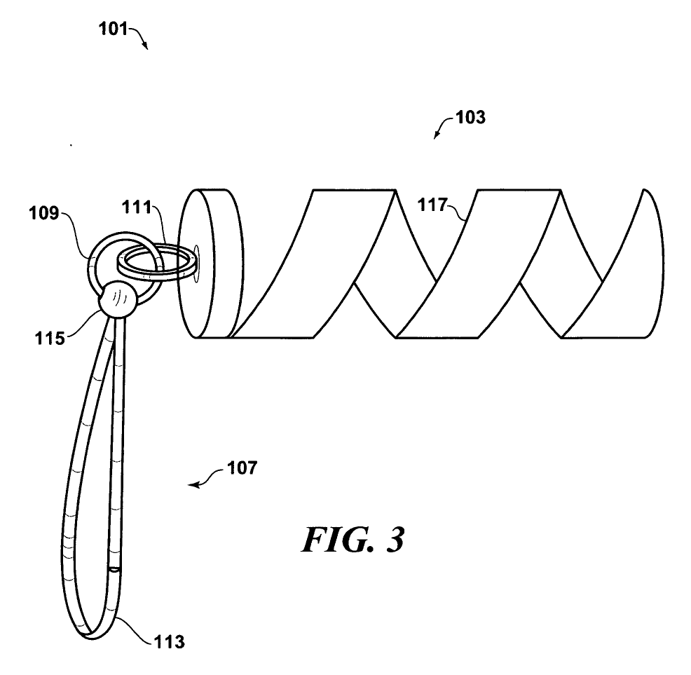

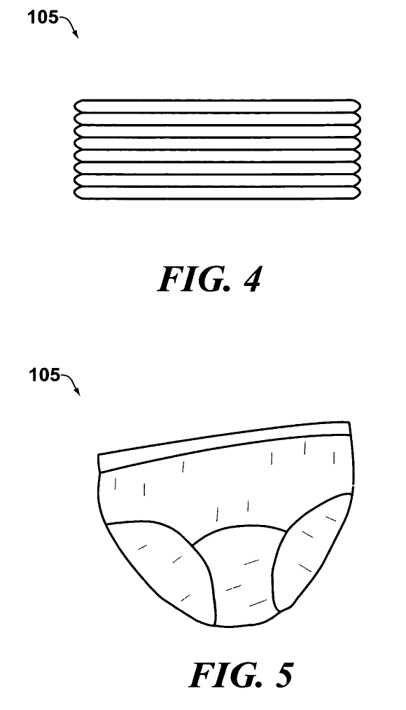

FIG. 3 depicts the device of FIG. 1 after the frangible seal 117 has been broken. This may be accomplished, for example, by banging the container 103 against the edge of a countertop or another suitable surface, or by twisting the container 103 with sufficient force. As seen therein, the pressurized container 103 then delaminates along the seal 117 so that its contents may be removed. FIG. 4 depicts the disposable undergarment 105 in a compact form as it might exist while it is disposed inside of the container 103, and FIG. 5 depicts the disposable undergarment 105 after it has been removed from the container 103 and unfolded.

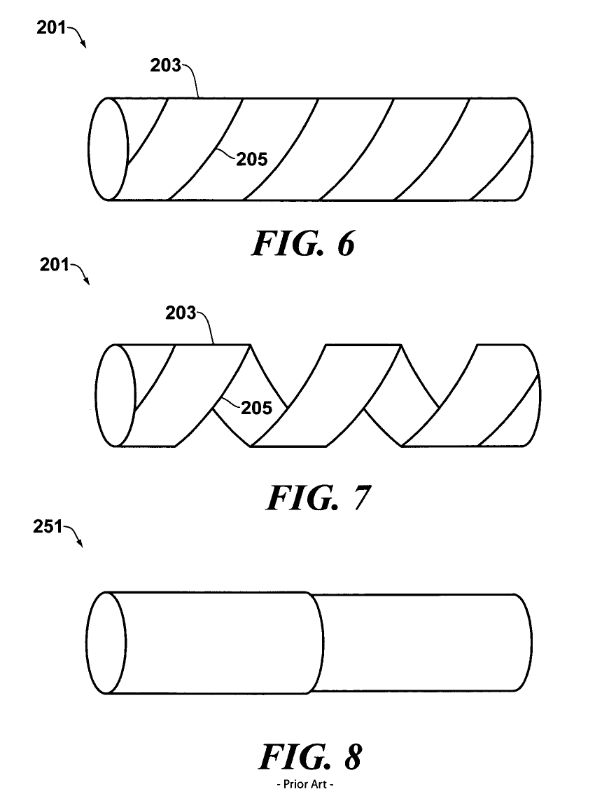

FIG. 6 depicts a second particular, non-limiting embodiment of a device in accordance with the teachings herein. This device 201 is similar in many respects to the first embodiment described above, but is styled to be of similar size and overall shape to a conventional tampon 251 of the type shown in FIG. 5. As with the previously described embodiment, this device 201 comprises a pressurized container 203 which is equipped with a frangible seal 205. FIG. 7 depicts the device 201 after the frangible seal 205 has been broken and the disposable undergarment (not shown) removed.

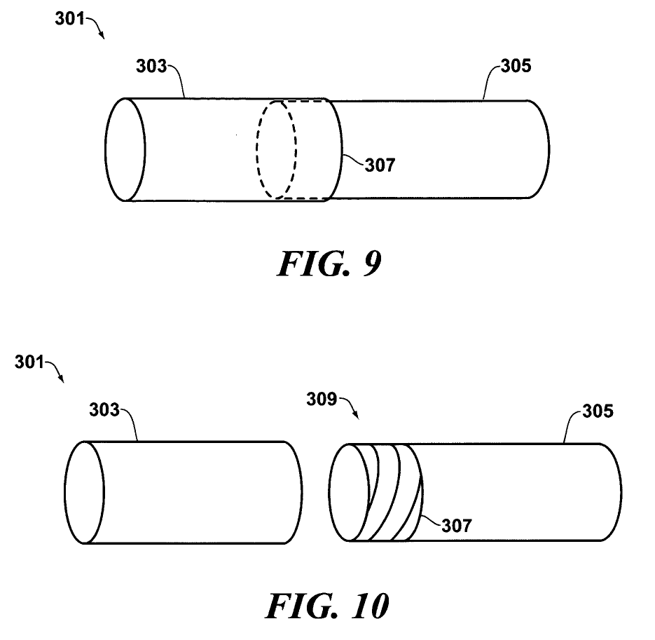

FIG. 9 depicts a third particular, non-limiting embodiment of a device in accordance with the teachings herein. This device 301 is similar in appearance to the conventional tampon 251 illustrated in FIG. 8, and is equipped with an upper portion 303 and a lower portion 305 which are joined together in overlapping fashion by way of a frangible seal 307. The frangible seal 307 in this particular embodiment is in the form of a portion of adhesive which is applied to one end of the lower portion 305 before the lower portion 305 is inserted into the upper portion 303. The upper 303 and lower 305 portions are preferably pressurized and thus separate from each other when sufficient torque is applied across the seal, thereby allowing the undergarment (not shown) which is housed therein to be removed.

The various containers utilized in the embodiments described above are generally cylindrical in shape, although one skilled in the art will appreciate that containers of various other geometries may also be utilized. Thus, for example, in various embodiments, the container may be polygonal (including, but not limited to, rectangular, square, hexagonal, or octagonal), elliptical, trapezoidal, or irregular in shape in a cross-section taken along its longitudinal axis.

The container is preferably charged with a portion of gas such that the interior of the container maintains a positive pressure with respect to the outside atmosphere (that is, so that the pressure within the container exceeds 760 torr (100 MPa) at sea level). Various gases may be used for this purpose including, for example, air or carbon dioxide. Preferably, however, an inert gas, such as nitrogen, argon or helium, is utilized. Though not preferred, in some embodiments, various hydrocarbon gases, fluorocarbon gases, hydrofluorcarbon gases, or hydrochlorocarbon gases may also be utilized. Of course, one skilled in the art will appreciate that various mixtures of the foregoing gases may also be employed.

Preferably, the container is constructed such that it is impermeable to the particular gas or gas mixture selected. This may be accomplished, for example, by constructing the container 103 out of materials which are inherently impermeable to the particular gas or gas mixture selected, or by treating the interior surface or walls of the container 103 with a material that renders them impermeable to the selected gas or gas mixture.

In the preferred embodiment, the container 103, or a portion thereof, may be sufficiently frangible such that the seal of the container may be broken by, for example, striking the container against the edge of the countertop or another suitable surface, or by bending or twisting the container with sufficient force. This may be accomplished, for example, by constructing the container 103 out of one or more helical strips of a gas-impermeable material such as paper, cardboard or another suitable material (these materials may be treated, if necessary, with resins, hydrocarbon waxes, or other suitable compositions to render them gas-impermeable, and are preferably recyclable or biodegradable), and by sealing the strips together with a suitable adhesive. Preferably, the strips are adhered together such that the resulting joints are somewhat weaker than the material of the strips themselves. Consequently, when the container is struck against a surface with sufficient force, the container will tend to rupture along these joints, after which the contents may be removed.

Through appropriate choice of adhesive, gas pressure and the materials out of which the container 103 is fabricated, the joints may be made to rupture with a maximum force that is sufficiently small to permit the seal of the container to be readily broken. Preferably, however, the minimum force is required to break the seal is sufficiently high to prevent accidental rupture of the container 103 due to normal handling thereof.

In some embodiments, various implements may be provided to facilitate rupturing the seal of the container. These implements may be provided in addition to, or in lieu of, the means described above. Thus, for example, the container may be provided with a pull tab, similar to the pull tabs currently utilized in tennis ball containers or soft drink cans, which is attached to a surface that is frangibly connected to the rest of the container. The container may also be provided with a pull string which breaks the seal as it is pulled on or unwound by the user.

The interior of the container may be charged with a suitable amount of perfume or other scented material to impart a desirable fragrance to the undergarments stored therein. Such a fragrance contributes favorably to the experience of using the product, while also imparting a feeling of cleanliness to the user. Such a fragrance may be in the liquid or gas state, or may comprise solid materials such as talcum powder. The interior of the container may also be charged with an antibacterial agent or the like.

The exterior of the container may be provided with various decorations, sealants, designs, indicia, or the like to impart a suitable aesthetic effect to the product. Preferably, however, the design of the container is not indicative of the contents thereof. This makes the product less embarrassing for the user to purchase and to carry on their person or store in a readily accessible place. In some embodiments, the manufacturer of the container may sell the advertising space provided on the surface of the container to various third parties for the advertisement of products unrelated to disposable undergarments.

As illustrated by the “key chain” embodiment depicted in FIGS. 1-3, devices may be made in accordance with the teachings herein which may be adapted to be permanently or removably attached to other articles or surfaces. Likewise, as illustrated by the embodiments depicted in FIGS. 6-7 and 9-10, these devices may also be constructed as stand-alone which can be carried in a purse or brief case or stored in a glove compartment, or which can readily fit into a pocket. In some embodiments, the container may be equipped with a repositionable adhesive, a releasable fastener (including hook and loop type fasteners of the type currently sold under the trade name VELCRO®) or other such features so that the product may be releasably secured to a suitable surface or article.

It will also be appreciated that the undergarments stored within the container may be compacted in various ways. The most optimal way may depend on a variety of factors, including the geometry of the container and the design and material of the undergarment. Thus, for example, the undergarment may be folded in accordion-type fashion in the manner depicted in FIG. 4, or may be wound into a compacted mass. In some cases, pneumatic presses or other devices may be utilized to compact the undergarments and to remove air pockets therefrom prior to their insertion into the container.

The undergarments may be inserted into the container in a variety of ways. For example, the undergarments may be compressed within a vacuum chamber (possibly using a pneumatic press) and may be placed into the container within the same vacuum chamber. In some embodiments, as wherein it is desired to vacuum pack the undergarment within the container, the container may then be sealed. In other more preferred embodiments, however, the container may subsequently be charged with a positive pressure of gas, and then sealed. Preferably, the undergarments are inserted into the container under hygienic or antiseptic conditions.

It will be appreciated that the devices described herein may be utilized with various undergarments, including panties, briefs (both male and female), garters, stockings, boxer shorts, jock straps, and the like. These devices may also be used in conjunction with various other articles including, but not limited to, hospital gowns, bibs, diapers, t-shirts, scarves, aprons, hair nets, smocks, costumes, veils, turbans, hand towels, and the like. Preferably, these articles of clothing are disposable and recyclable or biodegradable.

The devices described herein may be available over-the-counter, or may be dispensed from vending machines located in airports, bathrooms, schools, hospitals, business centers, or other places where a need for the product may arise.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims.

What is claimed is:

1. A device, comprising:
a frangible, pressurized container; and
an undergarment disposed in said container.

2. The device of claim 1, wherein the undergarment is in a highly compressed state.

3. The device of claim 1, wherein the container is generally cylindrical and shape.

4. The device of claim 1, further comprising a keychain attached to an exterior surface of the container.

5. The device of claim 1, further comprising a plurality of undergarments disposed and said container.

6. The device of claim 1, wherein the exterior surface of the container is not indicative of the contents thereof.

7. The device of claim 1, wherein the interior of said container is charged with a fragrance.

8. The device of claim 1, wherein said container comprises cardboard.

9. The device of claim 1, wherein said container comprises at least one helical strip of material, and wherein the edges of said helical strip are secured together with an adhesive.

10. The device of claim 1, wherein said container is adapted to rupture upon application of only a moderate force.

11. The device of claim 1, wherein said container is charged with an inert gas.

12. The device of claim 11, wherein said container is constructed from a material which is impermeable to said gas.

13. The device of claim 1, wherein said undergarment is a disposable undergarment.

14. A device, comprising:
a pressurized container having a wall which includes at least one helical strip, wherein the edges of said at least one strip are secured together with a suitable adhesive to form an enclosed volume; and
an undergarment disposed and said container.

15. The device of claim 14, wherein the joints formed by abutting edges of the at least one strip are frangible.

16. The device of claim 14, wherein the joints formed by abutting edges of the at least one strip are sufficiently frangible such that the seal of the container may be ruptured by tapping the container against an edge of a countertop using reasonable force.

17. A method for making a device, comprising:
providing a container;
compressing an undergarment through the application of force;
placing the undergarment into the container; and
charging the container with a positive pressure of gas.

18. The method of claim 17, wherein the pressure within the container exceeds 760 torr (100 MPa) at sea level.

19. The method of claim 17, wherein the undergarment is a disposable undergarment.

20. The method of claim 17, wherein the garment is placed in the container within in a vacuum.

21. The method of claim 17, wherein the container is frangible.
